(19) United States
(12) Patent Application Publication
(10) Pub. No.: US 2007/0238789 A1
(43) Pub. Date: Oct. 11, 2007

(54) PREDNISOLONE ACETATE COMPOSITIONS

(76) Inventors: Chin-Ming Chang, Tustin, CA (US); James N. Chang, Newport Beach, CA (US); Peter G. Bakhit, Huntington Beach, CA (US)

Correspondence Address: ALLERGAN, INC., 2525 DUPONT DRIVE, T2-7H, IRVINE, CA, 92612-1599, US

(21) Appl. No.: 11/278,354

(22) Filed: Mar. 31, 2006

(51) Int. Cl. A61K 31/155

(52) U.S. Cl. 514/632

(57) ABSTRACT

Prednisolone acetate compositions, and methods, eye drops, and medicaments related thereto, are disclosed herein.

### DESCRIPTION OF THE INVENTION

A composition comprising from about 0.6% to about 1% prednisolone acetate and from about 5% to about 30% 2-hydroxypropyl-γ-cyclodextrin is disclosed herein.

A composition comprising from about 0.6% to about 1% prednisolone acetate, from about 5% to about 30% 2-hydroxypropyl-γ-cyclodextrin, and polyquarternium-1 is disclosed herein.

A composition comprising from about 0.6% to about 1% prednisolone acetate, from about 5% to about 30% 2-hydroxypropyl-γ-cyclodextrin, and from about 80 ppm to about 500 pmm benzalkonium chloride is disclosed herein.

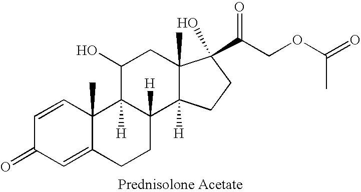

In one embodiment, the composition has about 0.6% prednisolone acetate.

In another embodiment the composition has about 1% prednisolone acetate.

2-Hydroxypropy-γ-cyclodextrin, CAS-No. 128446-34-4, is a γ-cyclodextrin derivative having the structure shown below. Its molecular weight is about 1500-1600, and has from about 0.5 to about 0.7 propylene oxide units per glucose unit. It is available from Wacker Fine Chemicals as CAVASOL® W8 HP Pharma.

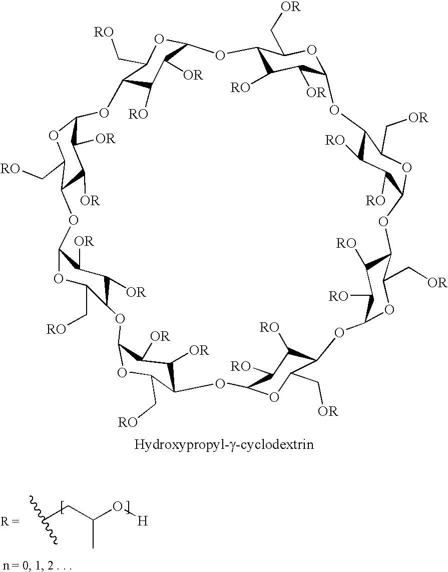

In one embodiment the composition has about 13% 2-hydroxypropyl-cyclodextrin.

In another embodiment the composition has about 21% 2-hydroxypropyl-cyclodextrin.

In another embodiment the composition has about 23% 2-hydroxypropyl-cyclodextrin.

A liquid which is suitable for topical ophthalmic administration is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions are often maintained at a comfortable pH with an appropriate buffer system. The formulations may also contain conventional, pharmaceutically acceptable preservatives, stabilizers and surfactants.

Various buffers and means for adjusting pH may be used so long as the resulting preparation is ophthalmically acceptable. Accordingly, buffers include, but are not limited to, acetate buffers, citrate buffers, phosphate buffers and borate buffers. Acids or bases may be used to adjust the pH of these formulations as needed. In one embodiment, the pH is from about 4 to about 5.

Benzalkonium chloride may be used as a preservative in the compositions disclosed herein. In one embodiment the concentration of benzalkonium chloride (BAK) is at least about 100 ppm. In another embodiment the concentration of benzalkonium chloride is at least about 150 ppm. In another embodiment the concentration of benzalkonium chloride is at least about 200 ppm. In another embodiment the concentration of benzalkonium chloride is at least about 300 ppm. In another embodiment the concentration of benzalkonium chloride is at least about 400 ppm. In another embodiment, the concentration of benzalkonium chloride is about 500 ppm or less.

Other useful preservatives include, but are not limited to, polyquartemium-1, chlorobutanol, thimerosal, phenylmercuric acetate and phenylmercuric nitrate.

A surfactant may be used for assisting in dissolving an excipient or an active agent, dispersing a solid or liquid in a composition, enhancing wetting, modifying drop size, or a number of other purposes. Useful surfactants, include, but are not limited to sorbitan esters, Polysorbate 20, Polysorbate 40, Polysorbate 60, Polysorbate 80, stearates, glyceryl stearate, isopropyl stearate, polyoxyl stearate, propylene glycol stearate, sucrose stearate, polyethylene glycol, polyethylene oxide, polypropylene oxide, polyethylene oxide-polypropylene oxide copolymers, alcohol ethoxylates, alkylphenol ethoxylates, alkyl glycosides, alkyl polyglycosides, fatty alcohols, phosphalipids, phosphatidyl chloline, phosphatidyl serine, and the like.

Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

In one embodiment, the composition comprises hydroxypropylmethylcellulose.

In another embodiment the composition has about 0.05% to about 0.15% hydroxpropylmethylcellulose.

In another embodiment the composition has about 0.1% hydroxypropylmethylcellulose.

Tonicity adjustors may be added as needed or convenient. They include, but are not limited to, salts, particularly sodium chloride, potassium chloride, mannitol and glycerin, or any other suitable ophthalmically acceptable tonicity adjustor.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. A useful chelating agent is edetate disodium (EDTA), although other chelating agents may also be used in place or in conjunction with it.

Unless otherwise indicated, all values of % are intended to mean % w/v at about 25° C.

### COMPOSITION EXAMPLES

The following are examples of compositions that are specifically contemplated herein.

### Composition Example 1

A composition comprising from about 0.6% to about 1% prednisolone acetate, from about 5% to about 30% 2-hydroxypropyl-γ-cyclodextrin, and from about 80 ppm to about 500 pmm benzalkonium chloride.

### Composition Example 2

The composition according to composition example 1 further comprising hydroxypropylmethylcellulose.

### Composition Example 3

The composition according to composition example 1 or 2 further comprising EDTA.

### Composition Example 4

The composition according to any one of composition examples 1 to 3 which further comprises sodium citrate.

### Composition Example 5

The composition according to any one of composition examples 1 to 4 wherein the pH is from about 4 to about 5.

### Composition Example 6

The composition according to any one of composition examples 1 to 6 having from about 100 ppm to about 500 ppm benzalkonium chloride.

### Composition Example 7

The composition according to composition example 6 having from about 150 ppm to about 500 ppm benzalkonium chloride.

### Composition Example 8

The composition according to composition example 7 having from about 200 ppm to about 500 ppm benzalkonium chloride.

### Composition Example 9

The composition according to composition example 8 having from about 300 ppm to about 500 ppm benzalkonium chloride.

### Composition Example 10

The composition according to composition example 9 having from about 400 ppm to about 500 ppm benzalkonium chloride.

### Composition Example 11

The composition according to any one of composition examples 1 to 10 having about 0.6% prednisolone acetate.

### Composition Example 12

The composition according to any one of composition examples 1 to 10 having about 1% prednisolone acetate.

### Composition Example 13

The composition according to any one of composition examples 1 to 12 having about 13% 2-hydroxypropyl-cyclodextrin.

### Composition Example 14

The composition according to any one of composition examples 1 to 12 having about 21% 2-hydroxypropyl-cyclodextrin.

### Composition Example 15

The composition according to any one of composition examples 1 to 12 having about 23% 2-hydroxypropyl-cyclodextrin.

### Composition Example 16

The composition according to any one of composition examples 2 to 15 having about 0.05% to about 0.15% hydroxpropylmethylcellulose.

### Composition Example 17

The composition according to any one of composition examples 2 to 16 having about 0.1% hydroxypropylmethylcellulose.

### Composition Example 18

The composition according to any one of composition examples 1 to 17 which is suitable for topical ophthalmic administration.

### Composition Example 19

A composition comprising from about 0.6% to about 1% prednisolone acetate and from about 5% to about 30% 2-hydroxypropyl-γ-cyclodextrin.

### Composition Example 20

The composition of composition example 19 further comprising polyquarternium-1.

### Eye Drop Example 1

An eye drop containing a composition according to any one of composition examples 1 to 18.

### Method Example 1

Use of a composition according to any one of composition examples 1 to 18 in the manufacture of a medicament for the treatment of an inflammatory condition in the eye of in a mammal.

### Method Example 2

A method comprising administering a composition according to any one of composition examples 1 to 18 to the surface of an eye of a mammal for the treatment of an inflammatory condition in the eye of said mammal.

### Kit example 1

A kit comprising a container for dispensing an eye drop of Eye Drop Example 1 and instructions for administration of said eye drop.

### EXPERIMENTAL EXAMPLES

Preservative Efficacy Tests

All test points represent N=1.

### Experimental Composition I

The composition below was prepared as follows:

To 70% of total purified water volume, add Cavasol W8 HP and dissolve by mixing.

Q.S. to 90% of volume with purified water.

Add Prednisolone Acetate and disperse.

Heat to 90° C. for 20 minutes or until Prednisolone Acetate is completely dissolved.

Cool to room temperature.

Add sodium citrate, dehydrate and dissolve.

Add EDTA and dissolve.

Add BAK and mix.

Adjust pH to 4.7 with 1N HCl.

Q.S to 100% of volume with purified water.

Sterile filter.

| Ingredient | % w/v |
|---|---|
| Prednisolone Acetate | 0.6% |
| Cavasol W8 HP | 23% |
| Sodium Citrate, Dihydrate | 0.05% |
| EDTA | 0.05% |
| BAK | See table below |
| pH | 4.7 |

The preservative efficacy data shown below was obtained for Experimental Composition I.

### Definitions

Marginal pass=1 to 1.8 log drop

Good pass=1.9 to 2.7 log drop

Solid pass=2.8 or higher log drop

USP Data is for full Panel at day 28.

| | Solution # | | | | | |
|---|---|---|---|---|---|---|
| | 1 | 2 | 3 | 4 | 5 | 6 |
| BAK (ppm) | 40 | 60 | 80 | 100 | 120 | 150 |
| USP Criteria | Fail | Fail | Fail | Marginal | Marginal | Good Pass |
| | | | | Pass | Pass | |

### Experimental Composition II

The composition below was prepared as follows:

Heat 70% of total purified water to 65°.

Add Hypromellose 2906 (HPMC F4M) and disperse.

Cool to hydrate and dissolve the Hypromellose.

Add Cavasol W8 HP and dissolve.

Add prednisolone acetate and disperse.

Q.S. to 95% of final volume.

Autoclave @ 120° C. for 20 minutes.

Remove while hot and stir overnight.

Add sodium citrate, dehydrate and dissolve.

Add EDTA and dissolve.

Add BAK and mix.

Adjust pH to 4.7 with 1N HCL.

Q.S. to final volume with purified water.

| Ingredient | % w/v |
|---|---|
| Prednisolone Acetate | 1.0% |
| Cavasol W8 HP | 21% |
| Hypromellose (HPMC F4M) | 0.1% |
| Sodium Citrate, Dihydrate | 0.05% |
| EDTA | 0.05% |
| BAK | See table below |
| pH | 4.7 |

USP Data is for full Panel at day 28.

| | Solution # | | | | | |
|---|---|---|---|---|---|---|
| | 1 | 2 | 3 | 4 | 5 | 6 |
| BAK (ppm) | 80 | 100 | 120 | 150 | 175 | 200 |
| USP Criteria | Fail | Fail | Marginal | Good | Good | Solid |
| | | | Pass | Pass | Pass | Pass |

### Experimental Composition III

The composition below was prepared as follows:

Heat 70% of total purified water to 65°.

Add Hypromellose 2906 (HPMC F4M) and disperse.

Cool to hydrate and dissolve the Hypromellose.

Add Cavasol W8 HP and dissolve.

Add prednisolone acetate and disperse.

Q.S. to 95% of final volume.

Autoclave at 120° C. for 20 minutes.

Remove while hot and stir overnight.

Add sodium citrate, dehydrate and dissolve.

Add EDTA and dissolve.

Add BAK and mix.

Adjust pH to 4.7 with 1N HCL.

Q.S. to final volume with purified water.

| Ingredient | % w/v |
|---|---|
| Prednisolone Acetate | 0.6% |
| Cavasol W8 HP | 13% |
| Hypromellose (HPMC F4M) | 0.1% |
| Sodium Citrate, Dihydrate | 0.1% |
| Sodium Chloride | 0.4% |
| EDTA | 0.05% |
| BAK | See table below |
| pH | 4.7 |

EP-B data is for bacteria only at 24 hours.
USP Data is for full Panel at day 14.

| | Solution # | | | | | | |
|---|---|---|---|---|---|---|---|
| | 1 | 2 | 3 | 4 | 5 | 6 | 7 |
| BAK | 50 | 75 | 100 | 150 | 200 | 250 | 300 |
| (ppm) | | | | | | | |
| EP-B | Fail | Fail | Fail | Fail | Fail | Fail | Fail |
| Criteria | | | | | | | |
| USP | Fail | Fail | Fail | Marginal | Good | Good | Not |
| Criteria | | | | Pass | Pass | Pass | Tested |

### Experimental Composition IV

The composition below was prepared as follows:

Heat 70% of total purified water to 65°.

Add Hypromellose 2906 (HPMC F4M) and disperse.

Cool to hydrate and dissolve the Hypromellose.

Add Cavasol W8 HP and dissolve.

Add prednisolone acetate and disperse.

Q.S. to 95% of final volume.

Autoclave at 120° C. for 20 minutes.

Remove while hot and stir overnight.

Add sodium citrate, dehydrate and dissolve.

Add EDTA and dissolve.

Add BAK and mix.

Adjust pH to 4.7 with 1N HCL.

Q.S. to final volume with purified water.

| Ingredient | % w/v |
|---|---|
| Prednisolone Acetate | 1.0% |
| Cavasol W8 HP | 21% |
| Hypromellose (HPMC F4M) | 0.1% |
| Sodium Citrate, Dihydrate | 0.1% |
| EDTA | 0.05% |
| BAK | See table below |
| pH | 4.7 |

EP-B data is for bacteria only at 24 hours.
USP Data is for full Panel at day 14.

| | Solution # | | | | | | | |
|---|---|---|---|---|---|---|---|---|
| | 1 | 2 | 3 | 4 | 5 | 6 | 7 | 8 |
| BAK | 150 | 200 | 250 | 300 | 350 | 400 | 450 | 500 |
| (ppm) | | | | | | | | |
| EP-B | Fail | Fail | Fail | Fail | Pass | Pass | Pass | Pass |
| Cri- | | | | | | | | |
| teria | | | | | | | | |
| USP | Good | Good | Solid | Not | Not | Not | Not | Not |
| Cri- | Pass | Pass | Pass | Test- | Test- | Test- | Test- | Test- |
| tera | | | | ed | ed | ed | ed | ed |

What is claimed is:

1. A composition comprising from about 0.6% to about 1% prednisolone acetate, from about 5% to about 30% 2-hydroxypropyl-γ-cyclodextrin, and from about 80 ppm to about 500 pmm benzalkonium chloride.

2. The composition according to claim 1 which is suitable for topical ophthalmic administration.

3. The composition according to claim 2 wherein the pH is from about 4 to about 5.

4. The composition according to claim 3 further comprising EDTA.

5. The composition according to claim 4 which further comprises sodium citrate.

6. The composition according to claim 5 further comprising hydroxypropylmethylcellulose.

7. The composition according to claim 1 having from about 100 ppm to about 500 ppm benzalkonium chloride.

8. The composition according to claim 7 having from about 150 ppm to about 500 ppm benzalkonium chloride.

9. The composition according to claim 8 having from about 200 ppm to about 500 ppm benzalkonium chloride.

10. The composition according to claim 9 having from about 300 ppm to about 500 ppm benzalkonium chloride.

11. The composition according to claim 10 having from about 400 ppm to about 500 ppm benzalkonium chloride.

12. The composition according to claim 3 about 0.6% prednisolone acetate.

13. The composition according to claim 3 having about 1% prednisolone acetate.

14. The composition according to claim 3 having about 13% 2-hydroxypropyl-cyclodextrin.

15. The composition according to claim 3 having about 21% 2-hydroxypropyl-cyclodextrin.

16. The composition according to claim 3 having about 23% 2-hydroxypropyl-cyclodextrin.

17. The composition according to claim 6 having about 0.05% to about 0.15% hydroxpropylmethylcellulose.

18. The composition according to claim 17 having about 0.1% hydroxypropylmethylcellulose.

19. A composition comprising from about 0.6% to about 1% prednisolone acetate and from about 5% to about 30% 2-hydroxypropyl-γ-cyclodextrin.

20. The composition of claim 19 further comprising polyquarternium-1.

21. A method comprising administering a composition according to claim 19 to the surface of an eye of a mammal for the treatment of an inflammatory condition in the eye of said mammal.
